SEQUENCE LISTING (5 sequences)

SEQ ID NO: 1 - protein, 184 aa, Homo sapiens [sequence omitted]

SEQ ID NO: 2 - DNA, 555 bp, Homo Sapiens
atgttgcagatggctgggcagtgctcccaaaatgaatattttgacagtttgttgcatgcttgcataccttgtcaacttcgatgttcttctaatactcctcctctaacatgtcagcgttattgtaatgcaagtgtgaccaattcagtgaaaggaacgaatgcgattctctggacctgtttgggactgagcttaataatttctttggcagttttcgtgctaatgtttttgctaaggaagataagctctgaaccattaaaggacgagtttaaaaacacaggatcaggtctcctgggcatggctaacattgacctggaaaagagcaggactggtgatgaaattatttctccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaa

SEQ ID NO: 3 - protein, 302 aa, Homo sapiens
METDTLLLWVLLLWVPGSTGDVTMLQMAGQCSQNEYFDSLLHACIPCQLRCSSNTPPLTCQRYCNASVTNSVKGVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 909 bp, Homo sapiens
atggagacagacacactcctgttatgggtgctgctgctctgggttccaggttccactggtgacgtcacgatgttgcagatggctgggcagtgctcccaaaatgaatattttgacagtttgttgcatgcttgcataccttgtcaacttcgatgttcttctaatactcctcctctaacatgtcagcgttattgtaatgcaagtgtgaccaattcagtgaaaggagtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 5 - protein, 21 aa, Mouse
MEVGWYRSPFSRVVHLYRNGK

(19) United States
(12) Patent Application Publication
(10) Pub. No.: US 2011/0177093 A1
(43) Pub. Date: Jul. 21, 2011

(54) USE OF BCMA AS AN IMMUNOREGULATORY AGENT

(75) Inventors: Susan L. Kalled, Concord, MA (US); Hugh Reid, Montmorency (AU)

(73) Assignees: Biogen, Inc.; Latrobe University

(21) Appl. No.: 12/862,448

(22) Filed: Aug. 24, 2010

Related U.S. Application Data
(63) Continuation of application No. 10/505,376, filed on Jul. 1, 2005, now abandoned.
(60) Provisional application No. 60/358,427, filed on Feb. 21, 2002.

(51) Int. Cl. A61K 39/395; A61P 37/02; A61P 25/00

(52) U.S. Cl. 424/158.1

(57) ABSTRACT

The disclosure relates to B-cell maturation antigen (BCMA), a receptor for APRIL and BAFF, and its use as an immunoregulatory agent in treatment of immunological disorders such as multiple sclerosis. The disclosure provides methods and compositions for treating neurodegenerative immunological disorders in mammals by administering soluble BCMA, an antibody against BCMA, or an antibody against a BCMA ligand, e.g., APRIL or BAFF.

This application claims priority to U.S. patent application Ser. No. 10/505,376, filed Jul. 1, 2005 (unintentially abandoned and pending a petition to revive under 37 CFR §1.137 (b)) and U.S. provisional patent application Ser. No. 60/358,427, filed Feb. 21, 2002, which is incorporated herein by reference in its entirety.

### TECHNICAL FIELD

The technical field of the invention relates to the use of B-cell maturation antigen (BCMA) as an immunoregulatory agent in treatment of immunological disorders such as multiple sclerosis.

### BACKGROUND OF THE INVENTION

Autoimmune diseases result from an abnormal immune response to self antigens. Generation of high affinity, somatically hypermutated auto-antibodies is one of the hallmarks of autoimmune conditions. Multiple sclerosis (MS) is an autoimmune disease with immune activity directed against central nervous system (CNS) antigens. An estimated 2,500,000 people in the world suffer from MS. MS is one of the most common diseases of the CNS in young adults. MS is a chronic, progressive, disabling disease, which generally strikes its victims at some time after adolescence, with diagnosis generally between 20 and 40 years of age, although onset may occur earlier. The disease is not directly hereditary, although genetic susceptibility plays a part in its development. MS presents in the form of recurrent attacks of focal or multifocal neurologic dysfunction. Attacks may occur, remit, and recur, seemingly randomly over many years. Remission is often incomplete and as one attack follows another, a stepwise downward progression ensues with increasing permanent deficit.

MS is a CD4⁺ T-cell-driven, neurodegenerative, chronic inflammatory disease. MS is associated with blood-brain barrier (BBB) dysfunction, infiltration of the CNS by mononuclear cells (mainly activated macrophages and T lymphocytes), demyelination (degeneration of the myelin sheaths surrounding axons) (Harris et al. (1991) Ann. Neurol., 29:548; Kermonde et al. (1990) Brain, 113:1477; Gironi et al. (2000) Neurol. Sci., 21(4 Suppl. 2):S871-5).

Although various immunotherapeutic drugs can provide relief in patients with MS, none is capable of reversing disease progression, and some can cause serious adverse effects. Therefore, the identification of compounds useful in prophylactically or therapeutically treating immune-mediated disorders, and particularly MS, is greatly needed. To understand and eventually counteract the pathogenic mechanisms in MS, a thorough knowledge of the immune response mediators is necessary. One approach to this problem is to test pathway inhibitors that affect different disease driving mechanisms in an animal model of MS.

Tumor necrosis factor (TNF) family members have been recognized as prominent regulators of the development and function of the immune system. Among other TNF family ligands, APRIL (a proliferation-inducing ligand) and BAFF (B cell activation factor) have been implicated in immune cell function. TACI (transmembrane activator and CAML-interactor) and BCMA (B cell maturation antigen) have been identified as cognate receptors for BAFF and APRIL, with each of the two receptors being able to bind either ligand. BCMA and TACI are expressed on B cells, whereas TACI is also expressed on activated T cells (Khare et al. (2001) Trends Immunol., 11:1547-1552). Treatment of mice with a soluble form of TACI or BCMA (TACI-Fc or BCMA-Fc) leads to reduced B cell numbers and a lack in the humoral response (Shu et al. (1999) J. Leukoc. Biol., 65:680-683; Yan et al. (2000) Nat. Immunol., 1:37-41; Xia et al. (200) J. Exp. Med., 192:137-143; Wang et al. (2001) Nat. Immunol., 2:632-637; Yu et al. (2000) Nat. Immunol., 1:252-256). For example, in a mouse model for rheumatoid arthritis, an autoimmune disease that involves both B and T cell components, TACI-Fc substantially inhibits inflammation and disease progression (Wang et al. (2001) Nat. Immunol., 2(7):632-637).

These effects are thought to be attributed to BAFF sequestration because: (1) BAFF was shown to act as a costimulator of B cells (Moore et al. (1999) Science, 285:260-263; Schneider et al. (1999) J. Exp. Med., 189:1747-1756; Mukhopadhyay (1999) et al. J. Biol. Chem., 274:15978-15981); (2) in vivo administration of a soluble form of BAFF results in splenomegaly due to increased B cell numbers (Moore et al. (1999) Science, 285:260-263); (3) transgenic (Tg) mice overexpressing BAFF display autoimmunity due to B cell expansion as a result of increased survival of normally deleted B cells (Khare et al. (2000) Proc. Natl. Acad. Sci., 97:3370-3375; Gross et al. (2000) Nature, 404:995-999; Mackay et al. (1999) J. Exp. Med., 190:1697-1710); and (4) BAFF-deficient mice have a phenotype similar to that of TACI-Fc- or BCMA-Fc-treated mice (almost complete loss of mature B cells and a severely compromised humoral response) (Schiemann et al. (2001) Science, 293:2111-2114; Gross et al. (2001) Immunity, 15:289-302). Based on this observation, it has been suggested that BAFF binding to TACI and/or BCMA may be essential for B cell survival and function. Nevertheless, TACI knockout (KO) mice show B cell expansion rather than death, while BCMA KO mice have normal phenotype (Schiemann et al. (2001) Science, 293:2111-2114; von Bulow et al. (2001) Immunity, 14:573-582; Xu et al. (2001) Nat. Immunol., 2:638-643), suggesting that BCMA's role is redundant. Despite the B cell expansion, T cell-dependent humoral response has been reported to be normal in the TACI KO mice (von Bulow et al. (2001) Immunity, 14:573-582). A significant decrease is observed, however, in responses to T cell-independent type 2 antigens (von Bulow et al. (2001) Immunity, 14:573-582; Yan et al. (2001) Nat. Immunol., 2:638-643). The recently identified third receptor for BAFF, BAFF-R, has helped reconcile at least some of these apparently contradictory reports. This receptor appears to mediate most of the B cell survival signal elicited by BAFF and explains to a large extent the phenotype of the BAFF Tg animals. In addition to a defect in B cell immunity, BAFF Tg mice have increased numbers of activated CD4⁺ and CD8⁺ T cells (Gross et al. (2000) Nature, 404:995-999; Mackay et al. (1999) J. Exp. Med., 190:1697-1710). Although these effects have not been characterized to the same extent as have the changes in B cell homeostasis, they do point to a possible role for BAFF in T cell activation. BAFF was also recently reported to provide costimulation for suboptimally activated T cells (Way et al. (2001) Nat. Immunol., 1:252-256).

The role of APRIL in immune regulation is less well understood. APRIL was originally described to stimulate growth of tumor cells in vitro and in vivo (Hahne et al. (1998) J. Exp. Med., 188:1185-1190). Recent reports demonstrated that APRIL may act as a costimulator of primary B and T cells in vitro and stimulate IgM production by peripheral blood B cells in vitro (Yu et al. (2000) Nat. Immunol., 1:252-256; Marsters et al. (2000) Curr. Biol., 10:785-788). Similar to BAFF, in vivo administration of APRIL results in splenomegaly due to expansion of the B cell population and an increase in the percentage of activated T cells (Yu et al. (2000) Nat. Immunol., 1:252-256), suggesting that APRIL plays a role in lymphoid homeostasis.

Therefore, there is a need in the art to understand the ultimate causes of various autoimmune disorders, and in particular MS, and to develop new therapeutic methods for treating and preventing such disorders.

### SUMMARY OF THE INVENTION

It is one of the objects of the present invention to provide methods and compositions for treating or preventing autoimmune disorders, such as MS, which are characterized by or associated with a risk of diminution of neurologic function. Additional objects of the invention will be set forth in part in the following description, and in part will be understood from the description, or may be learned by practice of the invention.

The present invention is based, in part, on the discovery and demonstration that in the experimental autoimmune encephalitis (EAE) model of MS, treatment of animals by administration of BCMA-Fc is effective in delaying the onset of acute disease and/or decreasing its severity.

The present invention provides methods for treating, preventing, and reducing risk of occurrence of neurodegenereative autoimmune disorders in mammals. The disclosed methods include administering to a subject susceptible to, or afflicted with, a neurodegenerative immunological disorder a therapeutically effective amount of modified BCMA so as to maintain desirable levels of neurologic function as assessed by clinical manifestations. In some embodiments, the methods reduce the progression of demyelination, the level of antigen-specific T-cell activity, and/or the level of CNS-specific autoantibodies. The populations treated by the methods of the invention include but are not limited to patients suffering or are at risk for the development of a neurodegenerative immunological disorder, such as MS. In some embodiment, the populations are suffering or are at risk for the development of diabetes, which may be co-presented with a degenerative immunological disorder.

Methods of administration and compositions used in the methods of the inventions are provided. The invention also provides assays for identifying and/or testing efficacy of a therapeutic compound for treatment of MS and related pathologies.

In certain embodiments, the compositions used in the methods of the invention comprise BCMA derivatives such as soluble forms of BCMA or antibodies against BCMA or against BCMA ligands (e.g., APRIL and/or BAFF). In some embodiments, soluble forms of BCMA used in the methods of the invention comprise (a) a first amino acid sequence derived from the ligand-binding domain of BCMA and (b) a second amino acid sequence derived from the constant region of an antibody. The first amino acid sequence is derived from all or a portion of the BCMA extracellular domain and is capable of binding a BCMA ligand specifically. The amino acid sequence of a ligand-binding domain of human BCMA is set out in SEQ ID NO:1 amino acid 1 to about amino acid 50. In a particular embodiment, the extracellular domain comprises amino acids 8-41 of SEQ ID NO:1. In certain embodiments, the first amino acid sequence is identical to or is substantially identical to amino acids 24-74 of SEQ ID NO:3. In an illustrative embodiment, BCMA-Ig comprises a sequence as in SEQ ID NO:3.

In some embodiments, the methods of the invention comprise administration of nucleic acids or polypeptides encoded by such nucleic acids, where the nucleotide sequence of such nucleic acid is selected from: (a) a nucleotide sequence from about nucleotide 70 to about nucleotide 213 of SEQ ID NO:4; and (b) a nucleic acid that is at least 60, 80, 100, 120, or 140 nucleotides long and is capable of hybridizing to the nucleic acid of (a) under defined conditions; wherein the expression product of the nucleic acid is capable of specifically binding to APRIL and/or BAFF. In an illustrative embodiment, such nucleotide comprises a sequence substantially as in SEQ ID NO:4.

It is to be understood that both the foregoing general description and the following detailed description are exemplary and explanatory only and are not restrictive of the invention as claimed.

### BRIEF DESCRIPTION OF THE FIGURES

FIGS. 1A and 1B depict graphs showing clinical scores and percent incidence respectively following a prophylactic treatment (days 1-17) with BCMA-Fc (or no treatment and IgG controls) in PLP-induced EAE SJL mice.

FIGS. 2A and 2B depict graphs showing clinical scores and relapse rate respectively in treatment with BCMA-Fc (or no treatment and IgG controls) during ongoing disease (days 17-34) in PLP-induced EAE SJL mice.

FIG. 3 shows titration of rMOG-specific IgG activity in NOD/Lt mice. rMOG-specific IgG levels were tested by ELISA.

FIG. 4 shows rMOG-specific Ig isotypes present in NOD/Lt mice. Sera (1/500 dilution) taken from each sacrificed mouse was tested by ELISA.

FIG. 5 shows concentration of total IgG+IgM in NOD/Lt mice. Sera taken from mice at trial completion of treatment were analyzed by ELISA for non-specific IgG & IgM concentration.

FIG. 6A-6B show NOD/Lt lymphocyte proliferation to (a) rMOG and (b) anti-CD3 antibody.

FIG. 7 shows concentration of pro-inflammatory (Th1-; IL-2, IL-6, GM-CSF, INF-γ) and anti-inflammatory (Th2-; IL-4, TGF-β) cytokines produced by splenocytes from NOD/Lt mice in response to rMOG.

FIGS. 8A-8C show the effect of BCMA-Fc on splenocyte proliferation in vitro. The ability of splenocytes to proliferate when stimulated by rMOG, MOG₃₅₋₅₅, BTN, anti-CD3 antibody and ConA from (a) BCMA-Fc, (b) IgG, and (c) PBS treatment group was measured.

### BRIEF DESCRIPTION OF SEQUENCES

SEQ ID NO:1 is an amino acid sequence of human BCMA.

SEQ ID NO:2 is human cDNA of BCMA.

SEQ ID NO:3 is an amino acid sequence of an illustrative embodiment of BCMA-Fc. Amino acids 1-23 are derived from murine IgGκ signal sequence; amino acids 24-74 are derived from the extracellular domain of human BCMA; and amino acids 75-302 are derived from the Fc region of human Ig heavy chain.

SEQ ID NO:4 is a nucleotide sequence encoding SEQ ID NO:3.

SEQ ID NO:5 is an amino acid sequence of the MOG₃₅₋₅₅ peptide.

### DETAILED DESCRIPTION OF THE INVENTION

In order that the present invention is more readily understood, certain terms are defined herein. Additional definitions are set forth throughout the detailed description.

The term “antibody,” as used herein, refers to an immunoglobulin or a part thereof, and encompasses any polypeptide comprising an antigen-binding site regardless of the source, method of production, and other characteristics. The term includes but is not limited to polyclonal, monoclonal, monospecific, polyspecific, non-specific, humanized, single-chain, chimeric, synthetic, recombinant, hybrid, mutated, and CDR-grafted antibodies. The term “antigen-binding domain” refers to the part of an antibody molecule that comprises the area specifically binding to or complementary to a part or all of an antigen. Where an antigen is large, an antibody may only bind to a particular part of the antigen. The “epitope,” or “antigenic determinant” is a portion of an antigen molecule that is responsible for specific interactions with the antigen-binding domain of an antibody. An antigen-binding domain may be provided by one or more antibody variable domains (e.g., a so-called Fd antibody fragment consisting of a V_{H} domain). An antigen-binding domain comprises an antibody light chain variable region (V_{L}) and an antibody heavy chain variable region (V_{H}). The term “anti-BCMA ligand antibody,” or “antibody against BCMA ligand,” refers to any antibody that specifically binds to at least one epitope of at least one BCMA ligand, including but not limited to APRIL or BAFF. The terms “BCMA antibody” and “antibody against BCMA” refer to any antibody that specifically binds to at least one epitope of BCMA.

The term “biological activity” refers to a function or set of functions (or the effect to which the function is attributed to) performed by a molecule in a biological system, which may be in vivo or in vitro. Biological activity may be assessed by, for example, the effect on lymphocyte proliferaction, survival, and function (e.g., secretion of cytokines), expression of cluster of differentiation markers on cell surface, the effect on propagation of action potential in, e.g., sensory or motor nerves, the effect on CNS function, the effect on gene expression at the transcriptional, translational, or post-translational levels, or the effect on autoantibody production, etc.

The term “clinical manifestations of MS” include any of a number of clinically recognized symptoms of MS, including but not limited to loss of motor and sensory neuronal function, fatigue, visual disturbances, loss of coordination, lack of muscle strength, altered sensory perception (e.g., hearing loss), problems associated with speech or swallowing, loss of bladder control, loss of cognitive function, weakness, spasticity, or pain (e.g., facial pain, such as trimengial neuralgia and muscle pain).

The term “hybridization under defined conditions” refers to conditions for hybridization and washes under which nucleotide sequences that are significantly identical or homologous to each other remain bound to each other. The conditions are such that sequences, which are at least 50, 100, 150, 300, or more nucleotides long and at least about 70%, more preferably at least about 80%, even more preferably at least about 85-90% identical, remain bound to each other. Unless the stringency of hybridization conditions is specifically stated, the term “hybridization under defined conditions” refers to conditions in which sequences that are at least 50 nucleotides long and at least about 70% identical remain bound to each other. The percent identity can be determined as described in Altschul et al. (1997) Nucleic Acids Res., 25:3389-3402. Nonlimiting examples of low stringency and high stringency hybridization conditions are provided in subsequent sections.

The term “mammal” refers to any animal classified as such, including humans.

The term “neurodegenerative immunological disorder” refers to a disease or condition that involves dysregulated immune response resulting in neurological pathology. Such disorders are associated with abnormalities in immune cell function or activity and characterized by aberrant or abnormal immune response, including aberrant autoimmune response. Examples of neurodegenerative immunological disorders include but are not limited to MS and other immune and autoimmune disorders or diseases such as acute inflammatory demyelinating polyneuropathy (AIDP), acute Guillain-Barre syndrome (GBS), or polyneuritis), chronic inflammatory demyelinating polyneuritis (CIDP), myasthenia gravis (MG), Eaton Lambert Syndrome (ELS), and encephalomyelitis. These disorders may be co-presented with, and potentially aggravated by diabetes, including but not limited to insulin-dependent (type 1) diabetes mellitus (IDDM).

The terms “treatment,” “therapeutic method,” and their cognates refer to both therapeutic treatment and prophylactic/preventative measures. Those in need of treatment may include individuals already having a particular medical disorder as well as those who may ultimately acquire the disorder.

The terms “therapeutic compound” and “therapeutic,” as used herein, refer to any compound capable of ameliorating clinical manifestations of a disorder, or to produce a desired biological outcome.

The terms “therapeutically effective dose” and “therapeutically effective amount” refer to that amount of a compound that results in prevention or amelioration of symptoms in a patient or a desired biological outcome, e.g., improved neuronal function, delayed onset of MS reduced levels of autoantibodies, etc. The effective amount can be determined as described in the subsequent sections.

The terms “specific interaction,” “specifically binding,” and their cognates, mean that two molecules form a complex that is relatively stable under physiologic conditions. Specific binding is characterized by a high affinity and a low to moderate capacity. Nonspecific binding usually has a low affinity with a moderate to high capacity. Typically, the binding is considered specific when the affinity constant Kₐ is higher than 10⁶M⁻¹, or preferably higher than 10⁸M⁻¹. If necessary, nonspecific binding can be reduced without substantially affecting specific binding by varying the binding conditions. Such conditions are known in the art, and a skilled artisan using routine techniques can select appropriate conditions. The conditions are usually defined in terms of protein concentration, ionic strength of the solution, temperature, time allowed for binding, concentration of unrelated molecules (e.g., serum albumin, milk casein), etc.

The phrase “substantially identical” means that a relevant amino acid sequence is at least 70%, 75%, 80%, 85%, 90%, 95%, 97%, 98%, 99%, or 100% identical to a given sequence. By way of example, such sequences may be variants derived from various species, or they may be derived from the given sequence by truncation, deletion, amino acid substitution or addition. Percent identity between two amino acid sequences is determined by standard alignment algorithms such as, for example, Basic Local Alignment Tool (BLAST) described in Altschul et al. (1990) J. Mol. Biol., 215:403-410, the algorithm of Needleman et al. (1970) J. Mol. Biol., 48:444-453, or the algorithm of Meyers et al. (1988) Comput. Appl. Biosci., 4:11-17.

In one aspect, this invention relates to the use of BCMA and BCMA-related molecules for treating neurodegenerative immunological disorders, such as MS.

BCMA is 35 kDa glycolipid-anchored protein which belongs to the TNF receptor family. BCMA specifically binds both APRIL (also known as TALL-2 or zTNF2) and BAFF (also known as BLyS, THANK, TALL-1, or zTNF4). As used herein, the term “BCMA ligand” (plural or singular) refers to APRIL and/or BAFF, individually or both, unless otherwise specifically stated. Nucleotide and amino acid sequences of these molecules are known in the art and can be obtained from publicly available databases, (see, e.g., www.ncbi.nlm.nih.gov). For example, BCMA is described in Laabi et al. (1992) EMBO J., 11(11):3897-3904 (Accession No. S43486). APRIL (Accession No. 075888) is described in Yu et al. (2000) Nature Immunology, 1:252-256 (also describes BCMA) and Hahne et al. (1998) J. Exp. Med., 188(6):1185-1190. BAFF is described in Schneider et al. (1999) J. Exp. Med., 189:1697-1710 (Accession No. AAD25356) and PCT Publication WO99/12964.

The term “BCMA,” as used herein, or its derivatives such as “BCMA-Fc,” “BCMA-Ig,” “soluble BCMA,” etc., refer to a molecule comprising at least a portion of a polypeptide set out SEQ ID NO:1 or a variant thereof, wherein such as a portion is sufficient for specific binding to APRIL and/or BAFF. In general, such a portion corresponds to and/or is referred to as a ligand-binding domain of receptor. cDNA encoding human BCMA is set out in SEQ ID NO:2.

The present invention is based, in part, on the discovery and demonstration that in the experimental autoimmune encephalitis (EAE) model of MS, treatment of animals by administration of a soluble form of BCMA is effective in delaying the onset of acute disease and/or decreasing its severity.

The invention provides methods for treating, preventing, and reducing risk of occurrence of neurodegenerative autoimmune disorders.

In certain embodiments, compositions used in the methods of the invention comprise soluble BCMA and/or anti-BCMA ligand antibodies.

BCMA, its fragments or other derivatives may be used to generate antibodies that specifically bind BCMA, while BCMA ligand(s), their fragments or other derivatives, or analogs thereof, may be used to generate antibodies that specifically bind BAFF or APRIL, or both (similarly to BCMA). In one embodiment, the antibody is a fully human recombinantly produced antibody. In the production of antibodies, screening for the desired antibody can be accomplished by techniques known in the art, most commonly, by ELISA or FACS. Antibodies can be made, for example, by traditional hybridoma techniques (Kohler and Milstein (1975) Nature, 256:495-499), recombinant DNA methods (U.S. Pat. No. 4,816,567), or phage display techniques using antibody libraries (Clackson et al. (1991) Nature, 352:624-628; Marks et al. (1991) J. Mol. Biol., 222:581-597). For various other antibody production techniques, see, e.g., Antibodies: A Laboratory Manual, eds. Harlow et al., Cold Spring Harbor Laboratory, 1988. In some embodiments, antibodies used in the methods of the invention are directed against at least a part of a BCMA ligand that specifically interacts with BCMA. In other embodiments, antibodies are capable of inhibiting the binding of BCMA ligand(s) to BCMA, e.g., competitively.

In some embodiments, the methods involve a use of soluble forms of BCMA (e.g., BCMA-Ig fusion polypeptides) that bind BCMA ligands thereby sequestering the BCMA ligand(s) and suppressing their activity in vivo. In particular, the presently disclosed soluble forms of BCMA inhibit the endogenous BAFF and/or APRIL activity associated with production T and B cell function. In some embodiments, BCMA-Ig possesses pharmacokinetic properties that make it suitable for therapeutic use, e.g., sufficiently long circulatory half-life and/or acceptable protection from proteolytic degradation.

BCMA-Ig used in the methods of the invention comprise (a) a first amino acid sequence derived from the extracellular domain of BCMA and (b) a second amino acid sequence derived from the constant region of an antibody.

The first amino acid sequence is derived from all or a portion of the BCMA extracellular domain and is capable of binding BCMA ligands specifically. The amino acid sequence of a ligand-binding domain of human BCMA is set out in SEQ ID NO:1 from about amino acid (aa) 1 to about aa 50. In certain embodiments, the first amino acid sequence is identical to or is substantially identical to amino acids 24-74 of SEQ ID NO:3. The sequence can be truncated or mutated so long as such a sequence retains the ability to specifically bind a BCMA ligand. In some other embodiments, the first amino acid sequence comprises at least 20, 25, 30, 35, 40, 45, or 50 contiguous amino acids of SEQ ID NO:1. In an illustrative embodiment, BCMA-Ig comprises a sequence as in SEQ ID NO:3. In another embodiment, BCMA-Ig comprises residues 2-54 of SEQ ID NO:1 fused to the Fc portion of human IgG. Such BCMA-Ig can be produced using standard techniques and is also available commercially, e.g., from Oncogene Research Products, San Diego, Calif. (Cat. No. PF089).

The second amino acid sequence is derived from the constant region of an antibody, particularly the Fc portion, or is a mutation of such a sequence. In some embodiments, the second amino acid sequence is derived from the Fc portion of an IgG. In related embodiments, the Fc portion is derived from IgG that is IgG₁, IgG₄, or another IgG isotype. In an illustrative embodiment, the second amino acid sequence comprises a sequence from amino acid (aa) 75 to aa 302 of SEQ ID NO:3. The second amino acid sequence may comprise the Fc portion of human IgG₁, wherein the Fc is modified to minimize the effector function. Such modifications include changing specific amino acid residues that might alter an effector function such as Fc receptor binding (Lund et al. (1991) J. Immun., 147:2657-2662 and Morgan et al. (1995) Immunology, 86:319-324), or changing the species from which the constant region is derived. Antibodies may have mutations in the C_{H}2 region of the heavy chain that reduce effector function, i.e., Fc receptor binding and complement activation. For example, antibodies may have mutations such as those described in U.S. Pat. Nos. 5,624,821 and 5,648,260. In the IgG₁ or IgG₂ heavy chain, for example, such mutations may be made at amino acid residues corresponding to amino acids 234 and 237 in the full-length sequence of IgG₁ or IgG₂. Antibodies may also have mutations that stabilize the disulfide bond between the two heavy chains of an immunoglobulin, such as mutations in the hinge region of IgG₄, as disclosed in Angal et al. (1993) Mol. Immunol., 30:105-108.

In certain embodiments, the second amino acid sequence is linked to the C-terminus or the N-terminus of the first amino acid sequence, with or without being linked by a linker sequence. The exact length and sequence of the linker and its orientation relative to the linked sequences may vary. The linker may, for example, comprise one or more Gly-Ser. The linker may be 2, 10, 20, 30, or more amino acid long and is selected based on properties desired such as solubility, length and steric separation, immogenicity, etc. It will be understood by one of ordinary skill in the art that certain amino acids in a sequence of any protein may be substituted for other amino acids without adversely affecting the activity of the protein. It is thus contemplated that various changes may be made in the amino acid sequences of BCMA of the invention, or DNA sequences encoding therefor without appreciable loss of their biological activity or utility.

The use of derivatives and analogs related to BCMA are also within the scope of the present invention. In a specific embodiment, the derivative or analog is functionally active, i.e., capable of exhibiting one or more activities associated with a ligand-binding domain of the wild-type BCMA. Derivatives or analogs that retain this binding, or inhibit biological activity of a BCMA ligand, can be tested by procedures known in the art.

Derivatives of BCMA-Ig, antibodies against BCMA or BCMA ligands can be made by altering their amino acids sequences by substitutions, additions, and/or deletions/truncations that result in functionally equivalent molecules. Due to the degeneracy of nucleotide codons, other DNA sequences that encode substantially the same amino acid sequence may be used in the practice of the present invention. These include but are not limited to nucleotide sequences that are altered by the substitution of different codons that encode a functionally equivalent amino acid residue within the sequence, thus producing a “silent” change. For example, the nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Substitutes for an amino acid within the sequence may be selected from other members of the class to which the amino acid belongs (see Table 1).

The BCMA derivatives and analogs of the invention can be produced by various techniques well known in the art, including recombinant and synthetic methods (Maniatis (1990) Molecular Cloning, A Laboratory Manual, 2nd ed., Cold Spring Harbor Laboratory, Cold Spring Harbor, N.Y., and Bodansky et al. (1995) The Practice of Peptide Synthesis, 2nd ed., Spring Verlag, Berlin, Germany).

TABLE 1

| Original | Exemplary | Typical |
|---|---|---|
| Residues | Substitutions | Substitutions |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, 1,4-Diamino-butyric Acid, Gln, Asn | Arg |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, Norleucine | Leu |

In certain embodiments, additional fusions of any of BCMA-Ig of the invention to amino acid sequences derived from other proteins may be constructed for use in the methods of the invention. Desirable fusion sequences may be derived from proteins having biological activity different from that of BCMA, for example, cytokines, growth and differentiation factors, enzymes, hormones, other receptor components, etc. Also, BCMA-Ig may be chemically coupled, or conjugated, to other proteins and pharmaceutical agents. Such modifications may be designed to alter the pharmacokinetics and/or biodistribution of the resultant composition. The BCMA-Ig and antibodies of the invention may also be glycosylated, pegylated, or linked to another nonproteinaceous polymer, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337. The BCMA-Ig and antibodies may be chemically modified by covalent conjugation to a polymer to increase their circulating half-life, for example. Exemplary polymers, and methods to attach them to peptides, are also shown in U.S. Pat. Nos. 4,766,106; 4,179,337; 4,495,285; and 4,609,546.

The BCMA-Ig and antibodies used in the methods of the invention may also be tagged with a detectable or functional label. Detectable labels include radiolabels such as ¹³¹I or ⁹⁹Tc, which may be attached using conventional chemistry. Detectable labels further include enzyme labels, e.g., horseradish peroxidase or alkaline phosphatase and detectable moieties such as biotin or avidin.

In some embodiments, the methods of the invention comprise administration of nucleic acids or polypeptides encoded by such nucleic acids, where the nucleic acid comprises a nucleotide sequence selected from: (a) a nucleotide sequence from about nucleotide 70 to about nucleotide 213 of SEQ ID NO:4; and (b) a nucleic acid that is at least 60, 80, 100, 120, or 140 nucleotides long and is capable of hybridizing to the nucleic acid of (a) under defined conditions; and wherein the expression product of the nucleic acid is capable of specifically binding to APRIL and/or BAFF. In an illustrative embodiment, such nucleotide sequence comprises a sequence substantially as in SEQ ID NO:4. In one embodiment, the defined conditions are low stringency conditions. In another embodiment, the defined conditions are moderate stringency conditions. In yet another embodiment, the defined conditions are high stringency conditions.

Appropriate hybridization conditions can be selected by those skilled in the art with minimal experimentation as exemplified in Ausubel et al. (1995), Current Protocols in Molecular Biology, John Wiley & Sons, sections 2, 4, and 6. Additionally, stringent conditions are described in Sambrook et al. (1989) Molecular Cloning: A Laboratory Manual, 2nd ed., Cold Spring Harbor Press, chapters 7, 9, and 11. A nonlimiting example of defined conditions of low stringency is as follows. Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10^{6 32}P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. Other conditions of low stringency well known in the art may be used (e.g., as employed for cross-species hybridizations).

A nonlimiting example of defined conditions of high stringency is as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in the prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 minutes.

Systems for cloning and expression of a polypeptide in a variety of different host cells are well known. Suitable host cells include bacteria, mammalian cells, and yeast and baculovirus systems. Mammalian cell lines available in the art for expression of a heterologous polypeptide include Chinese hamster ovary cells, HeLa cells, baby hamster kidney cells, NSO mouse melanoma cells and many others. A common bacterial host is E. coli. For other cells suitable for producing, e.g., BCMA-Ig, see Gene Expression Systems, eds. Fernandez et al., Academic Press, 1999.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids or viral, e.g., phage, or phagemid, as appropriate. For further details see, e.g., Molecular Cloning: A Laboratory Manual, Sambrook et al., 2nd ed., Cold Spring Harbor Laboratory Press, 1989. Many known techniques and protocols for manipulation of nucleic acid, for example, in preparation of nucleic acid constructs, mutagenesis, sequencing, introduction of DNA into cells and gene expression, and analysis of proteins, are described in detail in Current Protocols in Molecular Biology, eds. Ausubel et al., 2nd ed., John Wiley & Sons, 1992.

Following expression, BCMA-Ig is isolated and/or purified. Specific BCMA-Ig and their encoding nucleic acid molecules and vectors according to the present invention may be obtained, isolated and/or purified, e.g., from their natural environment, in substantially pure or homogeneous form, or in the case of nucleic acid, free or substantially free of nucleic acid or genes origin other than the sequence encoding a polypeptide with the required function.

The invention provides methods for treatment or prevention of various diseases and disorders by administration of a therapeutic compound (“therapeutic”). Suitable therapeutics include but are not limited to: BCMA, analogs and derivatives (including fragments) thereof; nucleic acids encoding the BCMA proteins, analogs, or derivatives; BCMA antisense nucleic acids, BCMA antibodies, BCMA ligand antibodies, and other BCMA/BCMA ligand antagonists.

Examples of immunological disorders susceptible to treatment by the methods of the invention include but are not limited to MS and other immune and autoimmune disorders or diseases such as acute inflammatory demyelinating polyneuropathy (AIDP), acute Guillain-Barre syndrome (GBS), or polyneuritis), chronic inflammatory demyelinating polyneuritis (CIDP), myasthenia gravis (MG), Eaton Lambert Syndrome (ELS), and encephalomyelitis. These disorders may be co-presented with, and possibly aggravated by diabetes, including but not limited to insulin-dependent diabetes mellitus (IDDM; type I diabetes).

In specific embodiments, therapeutics of the invention are administered in: (1) diseases or disorders involving elevated (i.e., relative to normal or desired) levels of expression of APRIL or BAFF or elevated APRIL or BAFF activity, or (2) diseases or disorders where in vitro or in vivo assays indicate the utility of BCMA administration (even in patients that have normal levels of APRIL or BAFF). The elevated level of expression or activity can be readily detected using methods standard in the art (e.g., Western blot, immunoprecipitation followed by SDS-PAGE, immunocytochemistry, etc.) and/or hybridization assays (e.g., Northern assays, dot blots, in situ hybridization, RT-PCR, etc.).

In an illustrative embodiment, mice are injected intraperitoneally or intravenously for a 3 week period with various frequencies with about 1 μg to about 1 mg, preferably about 10 μg to about 500 μg, or more preferably about 50 μg to about 200 μg of BCMA-Ig or an Ig control. The effectiveness of a compound is assessed by at least one of the following: clinical manifestations, demyelination, nerve conduction, immune cell activity, etc.

One embodiment of the invention provides assay for identifying agents effective as therapeutics for treatment of autoimmune disorders, including neurodegenerative disorders. In this screening assay, a first binding mixture is formed by combining a BCMA-Ig fusion polypeptide and a BCMA ligand, e.g., BAFF; and the amount of binding in the first binding mixture (M₀) is measured. A second binding mixture is also formed by combining the BCMA-Ig fusion polypeptide, the ligand, and the compound or agent to be screened, and the amount of binding in the second binding mixture (M₁) is measured. The amounts of binding in the first and second binding mixtures are then compared, for example, by calculating the M₁/M₀ ratio. The compound or agent is considered useful in treating an autoimmune disease if a decrease in binding in the second binding mixture as compared to the first binding mixture is observed. The formulation and optimization of binding mixtures is within the level of skill in the art, such binding mixtures may also contain buffers and salts necessary to enhance or to optimize binding, and additional control assays may be included in the screening assay of the invention. Compounds found to reduce BCMA/BCMA ligand binding by at least about 10% (i.e., M₁/M₀<0.9), preferably greater than about 30% or may thus be identified and then, if desired, secondarily screened for the capacity to ameliorate an autoimmune disorder in other assays or animal models as described below. The strength of the binding between a receptor and ligand can be measured using, for example, an enzyme-linked immunoadsorption assay (ELISA), radio-immunoassay (RIA), surface plasmon resonance-based technology (e.g., Biacore), all of which are techniques well known in the art.

Similarly to the procedures described in Examples, a test compound may be further assayed in an animal model of MS, known as Experimental autoimmune encephalomyelitis (EAE) (Tuohy et al. (1988) J. Immunol., 141:1126-1130, Sobel et al. (1984) J. Immunol., 132:2393-2401, and Traugott, 1989 Cell Immunol., 119:114-129). Chronic relapsing EAE provides a well-established experimental model for testing agents that would be useful for treatment of MS. The mouse EAE is an induced autoimmune demyelinating disease with many similarities to human MS in its clinical manifestations. In both EAE and MS, clinical disease is associated with blood-brain barrier (BBB) dysfunction, infiltration of central nervous system by mononuclear cells (mainly macrophages and T lymphocytes, and serum products), and demyelination (Baker et al. (1990) J. Neuroimmunol., 28:261; Butter et al. (1991) J. Neurol. Sci., 104:9; Harris et al. (1991) Ann. Neurol., 29:548; Kermonde et al. (1990) Brain, 113:1477).

Clinical signs of MS and demyelinating pathology in EAE result from immunization with CNS myelin proteins or peptides (e.g., MBP, PLP, and MOG) under Th1 conditions (direct immunization model), or by adoptive transfer of CNS antigen-specific Th1 cells (adoptive transfer model) (Ben-Nun et al. (1981) Eur. J. Immunol., 11:195-199; Ando et al. (1989) Cell Immunol., 124:132-143; Zamvil et al. (1985) Nature, 317:355-358; Zamvil et al. (1990) Ann. Rev. Immunol., 8:579-621). For example, in the SJL mouse model of EAE, immunization with the CNS peptide PLP 139-151 or adoptive transfer of PLP-specific Th1 cells results in a disease course consisting of an acute phase with loss of tail tone on day 10 to day 12, followed by hind limb paralysis and CNS mononuclear cell infiltration (Tuohy et al. (1988) J. Immunol., 141: 1126-1130, Sobel et al. (1984) J. Immunol., 132:2393-2401, and Traugott (1989) Cell Immunol., 119:114-129). Resolution of clinical signs and recovery occurs on day 20 to day 25 and the animals may undergo several more relapses less severe than the initial phase. EAE has been used to evaluate new therapeutic approaches to T-cell-mediated autoimmune disease because of the clinical and histopathological similarities to the human demyelinating MS.

One of skill in the art will appreciate that a compound may be optionally tested in at least one additional animal model (see, generally, Immunologic Defects in Laboratory Animals, eds. Gershwin et al., Plenum Press, 1981), for example, such as the following: the SWR X NZB (SNF1) mouse model (Uner et al. (1998) J. Autoimmune Disease, 11(3):233-240), the KRN transgenic mouse (K/BxN) model (Ji et al. (1999) Immunol. Rev., 169:139); NZB X NZW (B/W) mice, a model for SLE (Riemekasten et al. (2001) Arthritis Rheum., 44(10):2435-2445); the NOD mouse model of diabetes (Baxter et al. (1991) Autoimmunity, 9(1):61-67), etc.).

In certain embodiments, compounds to be tested are proteinaceous compounds (i.e., they comprise amino acids linked by peptide bonds) such as, e.g., soluble forms of BCMA (e.g., BCMA-Ig), antibodies against a BCMA ligand; analogs, derivatives, and fragments thereof. In certain other embodiments, the compounds are nucleic acids encoding such proteinaceous and antisense nucleic acids (and complementary and homologous sequences thereof).

Preliminary doses as, for example, determined according to animal tests, and the scaling of dosages for human administration is performed according to art-accepted practices. Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture assays or animal models. Levels in plasma may be measured, for example, by ELISA or HPLC. The effects of any particular dosage can be monitored by a suitable bioassay. Examples of dosages are: about 0.1×IC₅₀, about 0.5×IC₅₀, about 1×IC₅₀, about 5×IC₅₀, 10×IC₅₀, about 50×IC₅₀, and about 100×IC₅₀.

The data obtained from the in vitro assays or animal studies can be used in formulating a range of dosage for use in humans. Therapeutically effective dosages achieved in one animal model can be converted for use in another animal, including humans, using conversion factors known in the art (see, e.g., Freireich et al. (1966) Cancer Chemother. Reports, 50(4):219-244 and Table 2 for Equivalent Surface Area Dosage Factors).

TABLE 2

| | To: | | | | |
|---|---|---|---|---|---|
| | Mouse | Rat | Monkey | Dog | Human |
| From: | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and sex, as well as the severity of the medical condition in the subject. The dosage may be determined by a physician and adjusted, as necessary, to suit observed effects of the treatment. For BCMA-Ig, the compositions are administered so at a dose approximately: from 1 μg/kg to 20 mg/kg, from 1 μg/kg to 10 mg/kg, from 1 μg/kg to 1 mg/kg, from 10 μg/kg to 1 mg/kg, from 10 μg/kg to 100 μg/kg, from 100 μg to 1 mg/kg, and from 500 μg/kg to 1 mg/kg. The compositions may be given as a bolus dose, to maximize the circulating levels for the greatest length of time after the dose. Continuous infusion may also be used after the bolus dose.

In some embodiments, compositions used in the methods of the invention further comprise a pharmaceutically acceptable excipient. As used herein, the phrase “pharmaceutically acceptable excipient” refers to any and all solvents, dispersion media, coatings, antibacterial and antifungal agents, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition is formulated to be compatible with its intended route of administration. Methods to accomplish the administration are known in the art. “Administration” is not limited to any particular delivery system and may include, without limitation, parenteral (including subcutaneous, intravenous, intramedullary, intraarticular, intramuscular, or intraperitoneal injection) rectal, topical, transdermal, or oral (for example, in capsules, suspensions, or tablets). Administration to an individual may occur in a single dose or in repeat administrations, and in any of a variety of physiologically acceptable salt forms, and/or with an acceptable pharmaceutical carrier and/or additive as part of a pharmaceutical composition (described earlier). Physiologically acceptable salt forms and standard pharmaceutical formulation techniques and excipients are well known to persons skilled in the art (see, e.g., Physician's Desk Reference (PDR) 2003, 57th ed., Medical Economics Company, 2002; and Remington: The Science and Practice of Pharmacy, eds. Gennado et al., 20th ed, Lippincott, Williams & Wilkins, 2000).

Administration of a therapeutic to an individual may also be by means of gene therapy, wherein a nucleic acid sequence encoding the antagonist is administered to the patient in vivo or to cells in vitro, which are then introduced into a patient, and the antagonist (e.g., antisense RNA) is produced by expression of the product encoded by the nucleic acid sequence. Nucleic acids encoding proteinaceous compound, such as the nucleic acids encoding all or a part of BCMA or their corresponding antisense nucleic acids, can be introduced to a cell within tissue, an organ, or an organism so that the encoded polypeptides can then be expressed. For specific protocols, see Morgan, Gene Therapy Protocols, 2nd ed., Humana Press, 2000. This methodology may also be useful, for example, in evaluating effects of proteinaceous APRIL and/or BAFF antagonists on individual tissues, organs, or cell types. In certain embodiments, nucleic acid encoding a proteinaceous such a proteinaceous inhibitor is linked to a cell type-specific expression control sequence. Those of skill in the art will recognize that specific polynucleotide sequences can be inserted into the viral or plasmid vectors that can be injected into a mammal systemically, or locally. Host cells may also be harvested, and a nucleic acid may be transfected into such cells ex vivo for subsequent reimplantation using methods known in the art. Nucleic acids may be also transfected into a single cell embryo to create a transgenic animal as described in Gene Expression Systems, Academic Press, eds. Fernandez et al., 1999.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

### EXAMPLES

### Example 1

### Treatment of PLP-Induced EAE with BCMA-Fc

Prophylactic efficacy and dose response for BCMA-Fc were evaluated in a PLP-induced mouse EAE model as follows. 80 female 8 weeks old SJL mice purchased from Jackson Laboratory (Bar Harbor, Me.) were randomly assigned to eight groups of ten. Animals were housed 5 animals per cage in specially designed cage rack systems providing approximately 40 air changes per hour. Food and water were provided ad libitum.

EAE was induced in the mice as follows. On day 0, mice were injected subcutaneously (into each rear flank and near the dorsal midline) altogether with 80 μg bovine proteolipid protein (PLP) peptide 139-151, 200 μg Mycobacterium tuberculosis H37 RA (Difco Laboratories, Detroit, Mich., Cat. No. 3114-33-8) in 100 μl of incomplete Freund's adjuvant (ICFA) (Difco Laboratories, Cat. No. 0639-60-6).

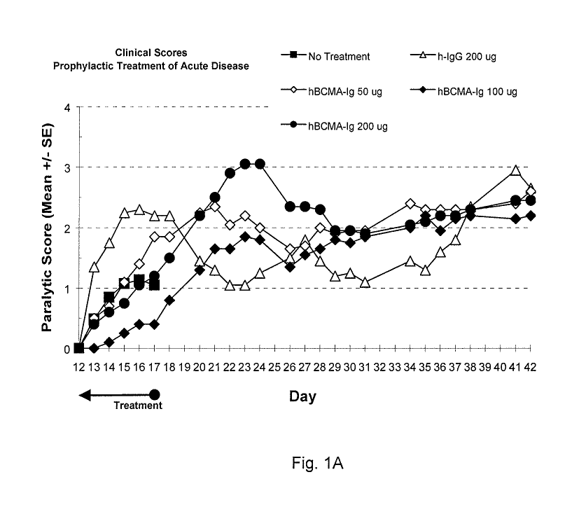

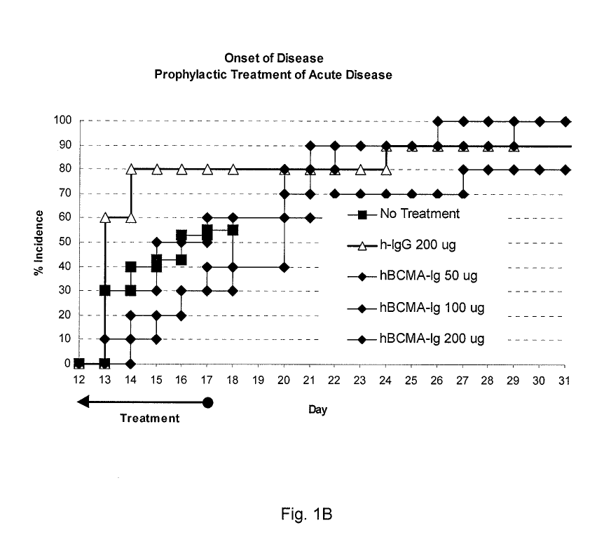

Beginning day 1 and through day 17, four groups of animals were injected IP with 200 μg of nonspecific polyclonal human IgG (Novartis; Basel, Switzeland) or with 50, 100, or 200 μg of human BCMA-Fc (Novartis). Beginning day 9, all animals were observed for signs of paralysis. Clinical signs were scored using a 5-point scale, where 0=normal; 1=limp tail or slight hind limb weakness; 2=hind limb weakness, 3=one hind limb paralyzed; 4=both hind limbs paralyzed; and 5=moribund or death. Remission was defined as at least 3 consecutive days of a score drop of at least 1, while relapse was defined as at least 3 consecutive days of a score increase of at least 1. The average disease score was determined for each group. Results are shown in FIGS. 1A and 1B and Table 3.

TABLE 3A
Prophylactic BCMA-Fc Treatment

| | | | Day | | | Effect on |
|---|---|---|---|---|---|---|
| | Onset | Peak | of | % | % | Righting |
| Group | Day | Score | Peak | Incidence | Mortality | Reflex |
| No Treatment | 14.00 | 2.45 | 15.14 | 55 | N/A | N/A |
| IgG 200 μg | 14.40 | 3.10 | 16.80 | 90 | 10 | 40 |
| BCMA-Fc | 17.30 | 2.90 | 20.10 | 100 | 20 | 30 |
| 50 μg | | | | | | |
| BCMA-Fc | 19.00 | 3.50 | 21.50 | 80 | 10 | 40 |
| 100 μg | | | | | | |
| BCMA-Fc | 18.80 | 3.30 | 21.10 | 100 | 10 | 10 |
| 200 μg | | | | | | |

The results indicate prophylactic treatment with BCMA-Fc delays the onset of acute disease and decreases the severity of resulting disease relapses when compared with the Ig control. Altough the treatment affected the righting reflex, this effect is not deemed to related to BCMA because it was the same in both control Ig or BCMA-Fc exhibited the same. The significance of this side effect in mice is unclear since a similar treatment with Ig in rat model of EAE did not produce this side effect.

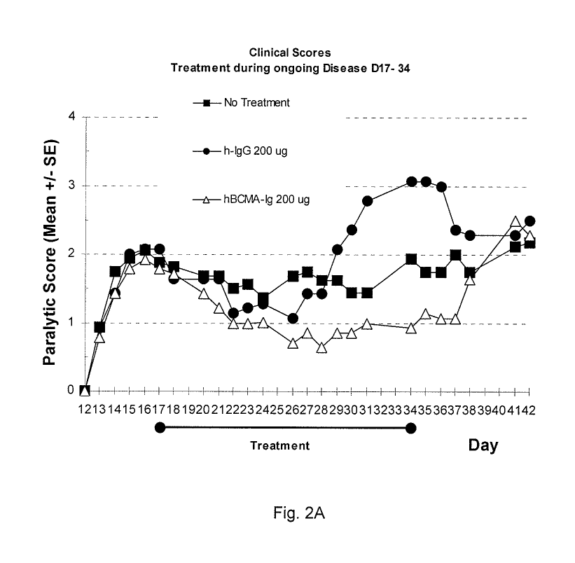

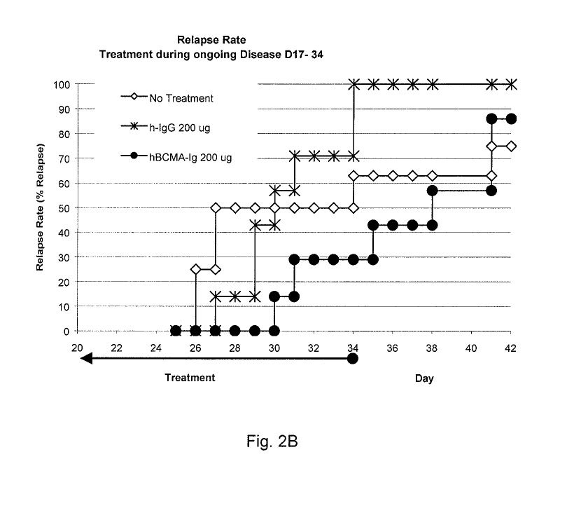

To determine the efficacy of treatment during ongoing disease mice from the no-treatment group were distributed into groups of 7-8 with normalized disease severity and administered 200 μg BCMA-Ig or 200 μg control Ig as described above beginning day 17 through day 34. Results are shown in FIGS. 2A and 2B and Table 3B.

TABLE 3B
Treatment During Ongoing Disease

| | | Day of | Max | | Effect on |
|---|---|---|---|---|---|
| | % Relapse | Relapse | Score of | % | Righting |
| | Incidence | Onset | Relapse | Mortality | Reflex |
| No Treatment | 75 | 30.2 | 2.8 | N/A | 0 |
| IgG 200 μg | 86 | 30.5 | 3.9 | 14 | 14 |
| BCMA-Ig 200 μg | 86 | 36.7 | 2.7 | 14 | 29 |

The results indicate that treatment during ongoing EAE with BCMA-Fc delays the onset and decreases the severity of the disease relapses when compared with the Ig control.

### Example 2

### Treatment MOG-Induced EAE with BCMA-Fc

Therapeutic efficacy of prophylactic treatment or during ongoing disease and dose response for BCMA-Fc were evaluated in a MOG-induced mouse EAE model as follows.

The extracellular domain of human myelin oligodendrocyte glycoprotein (MOG) (amino acid residues 1-121 of the mature protein) (rMOG) was produced in E. coli using the pQE9 expression vector (Qiagen, Australia) to incorporate an amino terminal histidine tag. rMOG was loaded onto a Ni-NTA Superflow (Qiagen, Australia) under denaturing conditions (8M urea) as per the manufacturer's instructions using a BioLogic LP Chromatography System (Bio-Rad Laboratories, Australia). The bound protein was washed with isopropanol to remove endotoxin, refolded on the column, eluted, and dialyzed against 50 mM NaCl in 10 mM Tris pH 8. Protein concentration and purity were estimated using a Micro BCA assay (Bio-Rad Laboratories, Australia) and SDS-PAGE, respectively. Endotoxin levels were determined using a Limulus Amebocyte Lysate assay (Associates of Cape Cod, Falmouth, Mass.).

Female 7-12 week old NOD/Lt mice (Animal Resource Center, Perth, Australia) were immunized subcutaneously with 100 μg of rMOG emulsified in complete Freund's adjuvant (CFA; Invitrogen, Australia) supplemented with Mycobacterium tuberculosis (4 mg/ml) (Difco Laboratories). Mice immediately received 350 ng of pertussis toxin (List Biological Laboratories, Campbell, Calif.) intravenously and again 48 hours later. Mice were monitored daily for clinical signs of neurological impairment and graded as follows: 0=no detectable impairment; 1=limp tail and loss of weight; 2=weakness of hind-limbs; 3=complete paralysis of one or both hind-limbs; 4=complete paralysis of one or both hind-limbs and ascending paralysis (at this stage mice were considered moribund and were euthanased). The individual scores from the day the treatment ensued were recorded, with the mean cumulative change in clinical score from each group on each subsequent day being determined. Mice were divided into three separate groups and, upon reaching a clinical score of approximately 2, treated intraperitoneally (IP) every second day for 55 days with either 100 μg of human BCMA-Fc (Biogen), 100 μg of nonspecific polyclonal human IgG (Biogen), or vehicle alone (phosphate buffered saline (PBS)). Experimental data was statistically analyzed using one-way ANOVA with either Tukey HSD or Scheffe post hoc analysis and Student's t-test. Results showing clinical response in mice treated upon disease onset (minimum clinical score of 2) are presented in Table 4. These results indicate that BCMA-Fc therapy significantly suppresses clinical symptoms and disease progression in mouse EAE.

TABLE 4
Treatment Upon Disease Onset

| Treatment | Mean^{a} clinical | | Mean | Number of mice that: | | |
|---|---|---|---|---|---|---|
| regimen | score at: | | change in | | | deceased |
| (number of | onset | end of | clinical | clinically | clinically | from |
| mice; n) | of treatment | treatment | score | improved^{b} | deteriorated^{c} | EAE |
| BCMA-Fc | 2.6 ± 0.18 | 2.1 ± 0.56 | −0.5 ± 0.39 | 2* | 2 | 1 |
| (n = 7) | | | | | | |
| IgG (n = 9) | 2.4 ± 0.13 | 3.1 ± 0.26 | +0.7 ± 0.22 | 1 | 6 | 1 |
| PBS (n = 8) | 2.4 ± 0.15 | 3.2 ± 0.14 | +0.8 ± 0.19 | 0 | 7 | 6 |

^{a}±standard error of the mean (SEM)
^{b}decreased clinical score at completion of treatment compared to onset of treatment
^{c}increased clinical score at completion of treatment compared to onset of treatment
*both mice made a full recovery to a clinical score of 0

### Example 3

### Effect of BCMA-Fc Treatment on Autoantibody Titers

Serum anti-MOG titer and isotype concentrations were determined by ELISA. 96-well microtiter plates were coated with rMOG (5 μg/ml), blocked, washed and incubated with test sera. Total Ig and isotypes bound were detected using Horse Radish Peroxidase (HRP)-coupled anti-Ig, IgG₁, IgG2c, IgG2b, IgG3, and IgM (Southern Biotechnology Associates, Inc., Birmingham, Ala.). Color was developed with ABTS (Sigma Aldrich, Australia) (450 ng/ml) and the optical density measured at 405 nm (OD₄₀₅) using a microplate reader (Molecular Devices, Sunnyvale, Calif.).

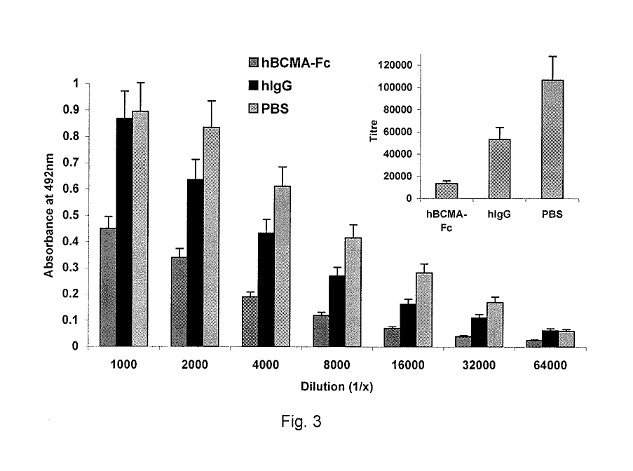

FIG. 3 shows titration of rMOG-specific IgG activity in NOD/Lt mice treated with BCMA-Fc, IgG, or PBS. Results at each dilution have been adjusted (subtraction of nonspecific binding) and represent mean comparable binding of each group ±SEM. The mean titer ±SEM for NOD/Lt mice was defined as the sera dilution giving an OD₄₉₂ three times higher than that of background.

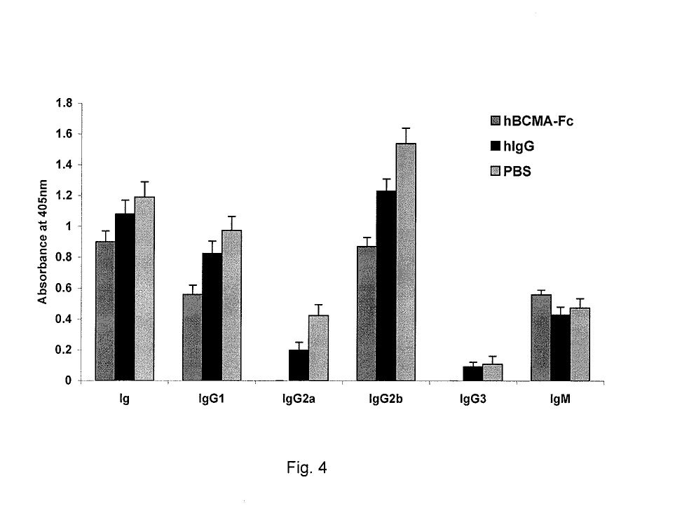

FIG. 4 shows rMOG-specific Ig isotypes present in NOD/Lt mice. Sera (1/500 dilution) taken from each sacrificed mouse was tested by ELISA. Results are expressed as mean OD₄₀₅ (nonspecific binding subtracted) ±SEM.

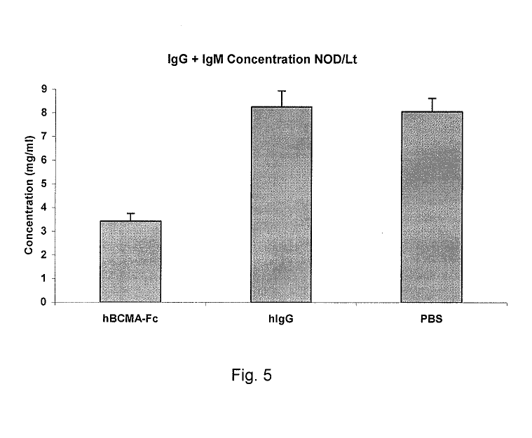

FIG. 5 shows concentration of total IgG+IgM in NOD/Lt mice. Sera taken from mice at trial completion were analyzed by ELISA for nonspecific IgG+IgM concentration. Results represent the mean concentration for each group ±SEM.

These results indicate that BCMA-Fc therapy dramatically reduces the titer of autoantibodies in mouse EAE.

### Example 4

### Effect of BCMA-Fc Treatment on T-Cell Responses

Spleens and lymph nodes were taken from NOD/Lt mice sacrificed at day 56 post-immunization. Single cell suspensions were prepared and cultured in 96-well tissue culture plates at 2×10⁵ cells/well in RPMI 1640 culture medium (Invitrogen, Australia) containing 5% heat-inactivated fetal calf serum (CSL, Melbourne, Australia), 20 μM β-mercaptoethanol, 2 mM L-glutamine, 1 mM sodium pyruvate, 10 mM HEPES, 100 U/ml penicillin, and 100 μg/ml of streptomycin (Invitrogen, Australia). Cells were cultured in the presence of 40 μl of different antigens: anti-CD3 (10 μg/ml), rMOG (20 μg/ml), mouse MOG₃₅₋₅₅ peptide (20 μg/ml; peptide sequence: SEQ ID NO:5; Auspep, Australia), recombinant butyrophilin first Ig domain (rBTN; produced as per rMOG) (20 μg/ml), Concanavalin A (Con A; 5 μg/ml) (Sigma Aldrich, Australia) and culture medium alone. Cell proliferation was measured by addition of [³H]-thymidine (1 μCi/well; Amersham Biosciences, Australia) for the last 16 hours of an 88-hour culture; [³H]-thymidine incorporation was determined by liquid scintillation counting (Wallac/PerkinElmer, Australia).

To investigate the effect of BCMA-Fc on splenocyte proliferation in vitro, 10 μg of BCMA-Fc was added to splenocyte cultures from pre-treated mice under the same conditions as described above. Controls using 10 μg of IgG and culture media alone were included. Cytokine profiles were gathered on supernatants of splenocyte cultures following 72 hour incubation with culture medium alone or in the presence of rMOG (20 μg/ml). IL-2, IL-4, IL-6, GM-CSF, TGF-β and IFN-γ concentrations were determined by ELISA, using capture and detection antibodies from Pharmingen (BD Biosciences, Australia) following the manufacturer's instructions. Recombinant mouse cytokines (Peprotech, Rocky Hills, N.J.) were used to generate standard curves.

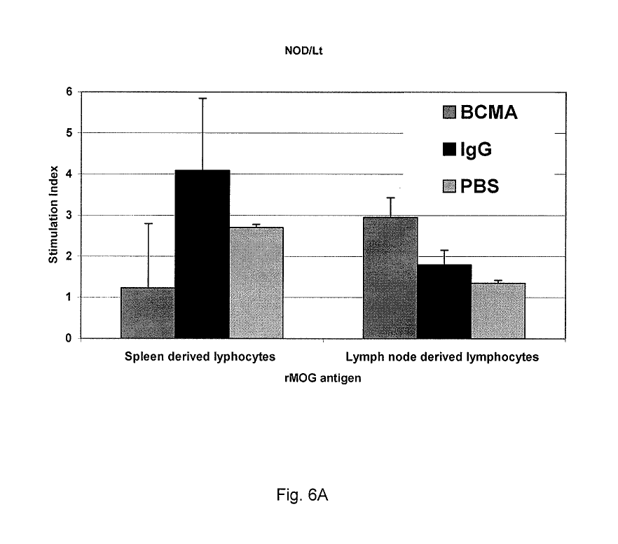

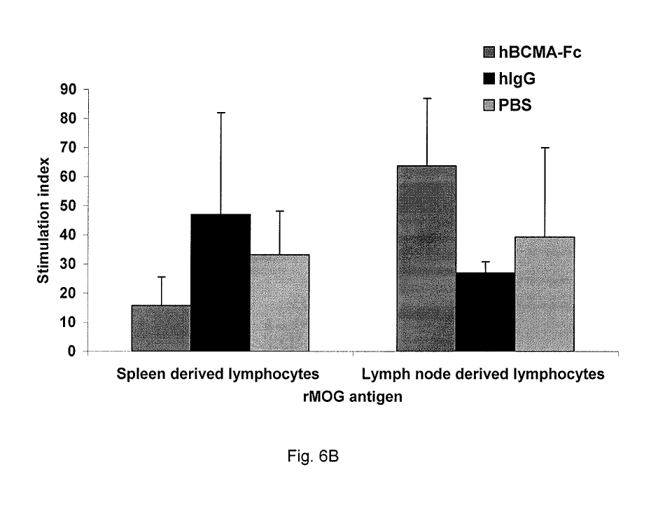

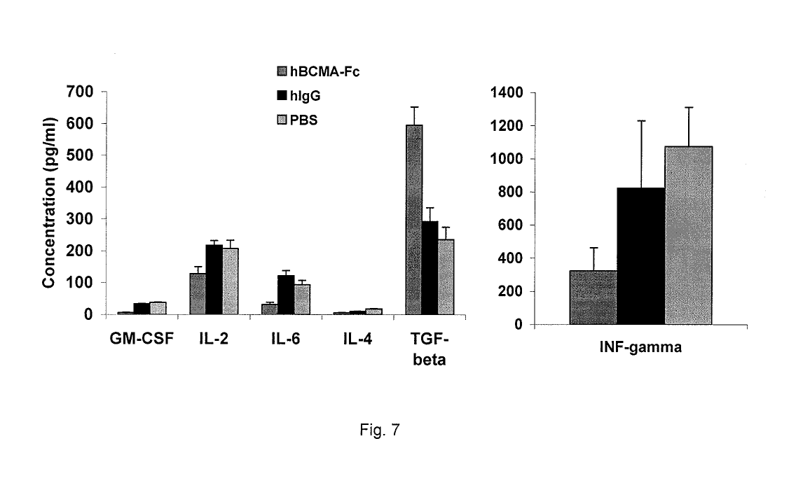

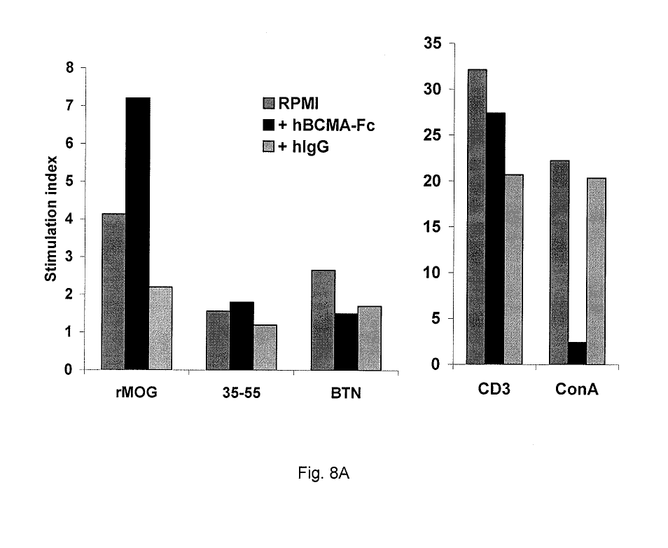

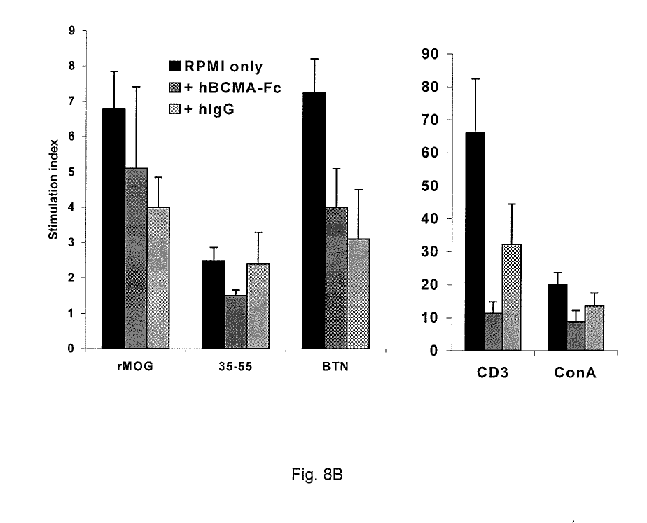

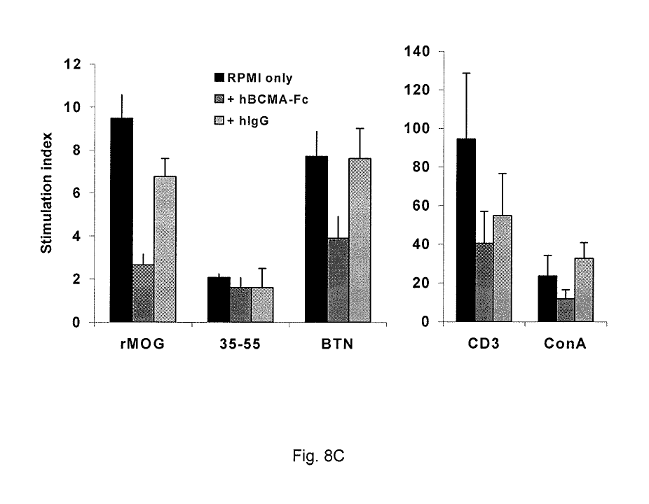

FIGS. 6A-6B show NOD/Lt lymphocyte proliferation to (a) rMOG and (b) anti-CD3 antibody. Bars represent the mean stimulation index (SI; mean counts per minute (cpm) in wells containing antigen (Ag) devided by mean cpm contain RPMI only (no Ag)) ±SEM. Mean cpm without Ag of spleen-derived and lymph node-derived lymphocytes respectively=1150 cpm; 948 cpm (BCMA-Fc; n=5), 1918 cpm; 1125 cpm (IgG, n=8), 1385 cpm; 845 cpm (PBS; n=2).

FIG. 7 shows concentration of pro-inflammatory (Th1-; IL-2, IL-6, GM-CSF, INF-γ) and anti-inflammatory (Th2-; IL-4, TGF-β) cytokines produced by splenocytes from NOD/Lt mice in response to rMOG. Results represent the mean concentration ±SEM.

FIGS. 8A-8C show the effect of BCMA-Fc on splenocyte proliferation in vitro. Splenocytes from mice in each treatment group were cultured in vitro in the presence of 10 μg/ml BCMA-Fc in RPMI. 10 μg/ml IgG and RPMI alone were used as controls. The ability of the cells to proliferate when stimulated by rMOG, MOG₃₅₋₅₅, BTN, anti-CD3 antibody & ConA from (a) BCMA-Fc (FIG. 8A), (b) IgG (FIG. 8B), and (c) PBS (FIG. 8C) treatment group was measured. Results represent the mean SI ±SEM. The mean cpm without Ag were almost identical for cultures containing 10 μg/ml BCMA-Fc, 10 μg/ml IgG, and RPMI alone.

For flow cytometry, single lymphocyte suspensions from spleen, lymph nodes, and peripheral blood were prepared. Whole blood was treated with 25 international units (IU)/ml of heparin (Sigma), plasma was removed and erythrocytes lysed in 5 ml of ACK lysis buffer for 10 min at RT, then washed and resuspended in PBS. For each tissue, 1×10⁶ cells in 50 μl of FACS staining buffer (BD Biosciences, Australia) were stained with 1 μg of fluorochrome-conjugated monoclonal antibodies to CD5, CD4, CD3, CD8, CD19, CD45R, CD138, or isotype controls (Pharmingen; BD Biosciences, Australia). Cell-associated fluorescence was analyzed on a FACScalibur flow cytometer using the Cellquest software (Becton Dickinson, San Jose, Calif.). The results of the FACS analysis for lymphocytes from peripheral blood (PB), spleen (SPL), or lymph nodes (LN) of NOD/Lt mice treated with BCMA-Fc every second day are presented in Tables 5A-5C, respectively. The results are expressed as mean percentage of B and T lymphocytes stained with a respective monoclonal antibody.

TABLE 5A
FACS Analysis on PB lymphocytes

| | BCMA-Fc | | IgG | | |
|---|---|---|---|---|---|
| | mean % ± SEM | n | mean % ± SEM | n | P-value |
| CD5−B220+ | 9.53 ± 1.11 | 4 | 13.68 ± 5.16 | 3 | — |
| CD19+CD8− | 7.99 ± 0.97 | 4 | 22.90 ± 5.11 | 6 | 0.04 |
| CD19+ | 8.67 ± 1.07 | 4 | 23.52 ± 5.09 | 6 | 0.029 |
| CD4+CD3+ | 31.32 ± 4.10 | 5 | 30.65 ± 5.77 | 3 | — |
| CD8+CD3+ | 26.42 ± 1.02 | 5 | 20.96 ± 2.83 | 3 | — |
| CD4+/CD8+ | 1.20 ± 0.18 | 5 | 1.41 ± 0.12 | 3 | — |

TABLE 5B
FACS Analysis on SPL lymphocytes

| | BCMA-Fc | | IgG | | |
|---|---|---|---|---|---|
| | mean % ± SEM | n | mean % ± SEM | n | P-value |
| B220+CD138+ | 2.21 ± 0.63 | 4 | 2.33 ± 0.67 | 3 | — |
| CD138+ | 2.94 ± 0.75 | 4 | 3.04 ± 0.89 | 3 | — |
| CD5−B220+ | 10.86 ± 3.38 | 3 | 22.90 ± 0.92 | 3 | 0.0117 |
| CD19+CD8− | 7.49 ± 1.99 | 2 | 30.44 ± 0.78 | 3 | 0.001 |
| CD19+ | 8.96 ± 0.95 | 2 | 31.04 ± 0.73 | 3 | 0.0003 |
| CD4+CD3+ | 48.15 ± 7.28 | 3 | 31.36 ± 4.50 | 3 | 0.117 |
| CD8+CD3+ | 18.89 ± 2.77 | 3 | 12.13 ± 2.03 | 3 | 0.119 |
| CD4+/CD8+ | 2.55 ± 0.01 | 3 | 2.61 ± 0.09 | 3 | — |

TABLE 5C
FACS Analysis on LN lymphocytes

| | BCMA-Fc | | IgG | | PBS | | IgG | PBS |
|---|---|---|---|---|---|---|---|---|
| | Mean % SEM | n | Mean % ± SEM | n | Mean % ± SEM | n | P-value | P-value |
| B220+CD138+ | 1.70 ± 1.07 | 6 | 2.33 ± 0.42 | 6 | 2.29 ± 0.39 | 2 | — | — |
| CD138+ | 2.45 ± 0.44 | 6 | 3.12 ± 1.42 | 6 | 3.12 ± 0.47 | 2 | — | — |
| CD5−B220+ | 2.67 ± 0.30 | 4 | 6.66 ± 0.82 | 3 | | | — | — |
| CD19+CD8− | 2.46 ± 0.50 | 6 | 15.37 ± 1.05 | 6 | 13.63 ± 3.59 | 2 | <0.002 | |
| CD19+ | 4.08 ± 0.53 | 6 | 16.57 ± 2.57 | 6 | 15.50 ± 3.60 | 2 | <0.0001 | 0.0012 |
| CD4+CD3+ | 61.31 ± 0.79 | 6 | 51.19 ± 2.51 | 6 | 53.39 ± 1.09 | 2 | <0.0001 | 0.0003 |
| CD8+CD3+ | 32.72 ± 1.03 | 6 | 24.89 ± 2.77 | 6 | 23.03 ± 1.76 | 2 | — | — |
| CD4+/CD8+ | 1.89 ± 0.08 | 6 | 2.07 ± 2.27 | 6 | 2.32 ± 0.13 | 2 | 0.0005 | 0.014 |

The results indicate that in addition to dramatically reducing the titer of autoantibodies (Example 4), BCMA-Fc also induces a switch in the subtype of the T helper-cell population characterized by marked alterations in cytokine production following restimulation in vitro with MOG. BCMA-Fc therapy leads to significant increases in the level of Th2/Th3 cytokines while the levels of Th1 cytokines are significantly diminished. Therefore, BCMA-Fc is able to effectively target both effector arms of the immune response in EAE.

### Example 5

### Effect of BCMA-Fc Treatment on CNS Pathophysiology

Upon CO₂ asphyxiation, brain and spinal cord from NOD/Lt mice were dissected, immersion fixed in 4% formalin and embedded in paraffin blocks. Blocks were cut in the caudal to rostral direction and adjacent sections stained with luxol fast blue (LFB) to stain for myelin or haematoxylin-eosin (H&E) to stain for inflammation. Stained sections were examined and scored using a scale set by the level of demyelination and perivascular infiltration/inflammatory lesions (where 0=no demyelination or inflammation; up to a maximum of 5=extensive demyelination or inflammation) using a conventional microscope while blind to the treatment regimen. Results of H&E staining in the forebrain, Pons medulla, cerebellum, and spinal cord (S.C.) is presented in Table 6A. Results of a corresponding histological analysis using LFB staining is presented Table 6B.

TABLE 6A
Effect of BCMA-Fc Treatment on Inflammation

| Mouse | | | | | | |
|---|---|---|---|---|---|---|
| Strain & | | Clinical | Fore- | Pons | | |
| No. | Treatment | Sore | brain | medulla | Cerebellum | S.C. |
| NOD 4 | PBS | 3 | 3 | 5 | 5 | 5 |
| NOD 8 | PBS | 3 | 1 | 1 | 4 | 1 |
| NOD 17 | IgG | 3 | 5 | 4 | 5 | 2 |
| NOD 27 | IgG | 3.5 | 0 | 1 | 1 | 5 |
| NOD 2 | BCMA-Fc | 3 | 0 | 0 | 1 | 2 |
| NOD 10 | BCMA-Fc | 3.5 | 0 | 0 | 0.5 | 1 |
| NOD 26 | BCMA-Fc | 0 | 0 | 0 | 1 | 2 |

TABLE 6B
Effect of BCMA-Fc Treatment on Demyelination

| Mouse | | | | | | |
|---|---|---|---|---|---|---|
| Strain & | | Clinical | Fore- | Pons | | |
| No. | Treatment | Sore | brain | medulla | Cerebellum | S.C. |
| NOD 4 | PBS | 3 | — | 5 | 5 | 2 |
| NOD 8 | PBS | 3 | — | 1 | 5 | 3 |
| NOD 17 | IgG | 3 | — | 2 | 3 | 2 |
| NOD 27 | IgG | 3.5 | — | 0 | 2 | 5 |
| NOD 2 | BCMA-Fc | 3 | — | 0 | 1.5 | 2 |
| NOD 10 | BCMA-Fc | 3.5 | — | 0 | 1 | 5 |
| NOD 26 | BCMA-Fc | 0 | — | 0 | 1 | 2 |

The histochemical results demonstrate that BCMA-Fc therapy significantly suppresses the clinical symptoms and disease progression and that such therapy corresponds with an inhibition of CNS inflammation and demyelination.

The specification is most thoroughly understood in light of the teachings of the references cited within the specification which are hereby incorporated by reference. The embodiments within the specification provide limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with the present specification, the present specification will supercede any such material. The citation of any references herein is as not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may very depending upon the desired properties sought to be obtained by the present invention. Those skilled in the art will recognize, or be able to ascertain using no more than routine experimentation, many equivalents to the specific embodiments of the invention described herein. Such equivalents are intended to be encompassed by the following claims.

1-23. (canceled)

24. A method of treating multiple sclerosis in a mammal, comprising administering to the mammal a therapeutically effective amount of an antibody to BAFF, thereby treating the multiple sclerosis.

25. A method of treating demyelination in a mammal, comprising administering to the mammal a therapeutically effective amount of an antibody to BAFF, thereby treating demyelination, wherein the mammal has, or is at risk for developing, multiple sclerosis.

26. A method of treating CNS inflammation in a mammal, comprising administering to the mammal a therapeutically effective amount of an antibody to BAFF, thereby treating CNS inflammation, wherein the mammal has, or is at risk for developing, multiple sclerosis.

27. A method of reducing a CNS-specific autoantibody titer in a mammal, comprising administering to the mammal a therapeutically effective amount of an antibody to BAFF, thereby reducing the CNS-specific autoantibody titer, wherein the mammal has, or is at risk for developing, multiple sclerosis.

28. The method of claim 24, wherein the mammal has, or is at risk for developing, diabetes.

29. The method of claim 25, wherein the mammal has, or is at risk for developing, diabetes.

30. The method of claim 26, wherein the mammal has, or is at risk for developing, diabetes.

31. The method of claim 27, wherein the mammal has, or is at risk for developing, diabetes.

32. The method of claim 24, wherein the mammal is human.

33. The method of claim 25, wherein the mammal is human.

34. The method of claim 26, wherein the mammal is human.

35. The method of claim 27, wherein the mammal is human.
